(19) United States
(12) Patent Application Publication
(10) Pub. No.: US 2015/0157279 A1
(43) Pub. Date: Jun. 11, 2015

(54) METHOD, COMPUTER-READABLE STORAGE DEVICE AND APPARATUS FOR PROVIDING AMBIENT AUGMENTED REMOTE MONITORING

(71) Applicants: President and Fellows of Harvard College, Cambridge, MA (US); AT&T Intellectual Property I, L.P., Atlanta, GA (US)

(72) Inventors: SAEED S. GHASSEMZADEH, Andover, NJ (US); Lusheng Ji, Randolph, NJ (US); Robert Raymond Miller, II, Convent Station, NJ (US); Manish Gupta, Cambridge, MA (US); Vahid Tarokh, Cambridge, MA (US)

(73) Assignees: President and Fellows of Harvard College, Cambridge, MA (US); AT&T Intellectual Property I, L.P., Atlanta, GA (US)

(21) Appl. No.: 14/099,610

(22) Filed: Dec. 6, 2013

(51) Int. Cl. A61B 5/00; A61B 5/0205; A61B 5/11; A61B 5/145; A61B 5/0402

(52) U.S. Cl. CPC A61B 5/746; A61B 5/7282; A61B 5/14532; A61B 5/0402; A61B 5/112; A61B 5/02055; A61B 5/6802; A61B 5/6887; A61B 5/021

(57) ABSTRACT

A method, computer-readable storage device, and an apparatus for processing an alert are disclosed. For example, the method receives the alert reporting a physiological condition for a individual, analyzes the physiological condition of the individual in view of ambient information in a vicinity of the individual, generates a report in accordance with the analyzing, and sends the report to a recipient.

### BACKGROUND

A health monitoring system may monitor the health of patients. The data obtained for a particular patient may be compared to either historical norms of the particular patient or general population norms. A need for an alert may then be assessed based on the results of the comparison. Unfortunately, the accuracy of the assessment may be biased due to changes in the patient's surroundings. Thus, the assessment of the need for the alert can be inaccurate.

### BRIEF DESCRIPTION OF THE DRAWINGS

The teaching of the present disclosure can be readily understood by considering the following detailed description in conjunction with the accompanying drawings, in which:

FIG. 1 illustrates an exemplary network related to the present disclosure;

FIG. 2. illustrates a flowchart of a method for providing an ambient augmented telehealth remote monitoring; and

FIG. 3 depicts a high-level block diagram of a general-purpose computer suitable for use in performing the functions described herein.

To facilitate understanding, identical reference numerals have been used, where possible, to designate identical elements that are common to the figures.

### DETAILED DESCRIPTION

The present disclosure broadly describes a method, computer-readable storage device and apparatus for providing an ambient augmented telehealth remote monitoring. Although the teachings of the present disclosure are discussed below in a context of a Generic Analytic Engine (GAE), the teaching is not so limited. Namely, the teachings of the present disclosure can be applied for other types of telehealth monitoring systems, wherein the analysis of the data may be improved based on knowledge of ambient information. In one embodiment, the present disclosure describes a method, computer-readable storage device, and an apparatus for processing an alert. For example, the method receives the alert reporting a physiological condition for a individual, analyzes the physiological condition of the individual in view of ambient information in a vicinity of the individual, generates a report in accordance with the analyzing, and sends the report to a recipient.

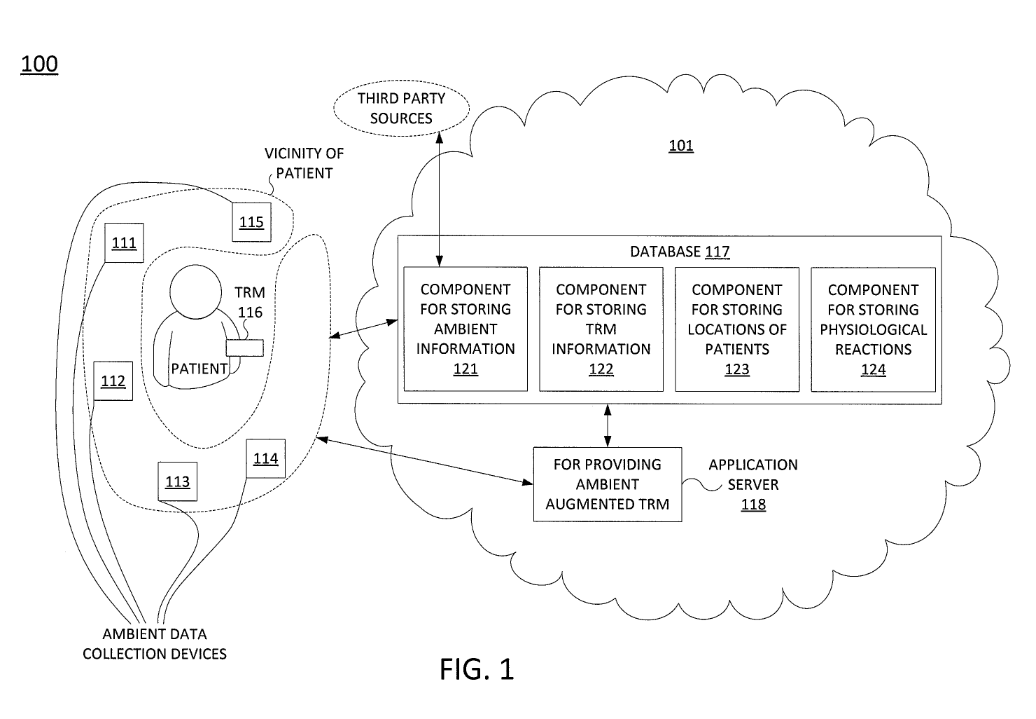

FIG. 1 illustrates an exemplary network 100 related to the present disclosure. In one illustrative embodiment, the network 100 comprises ambient data collection devices 111-115, a Telehealth Remote Monitoring (TRM) device (broadly a portable monitoring device) 116, a database 117, and an application server 118 for providing an ambient augmented TRM.

In one embodiment, application server 118 and database 117 are deployed in a communications network 101. For example, the communication network 100 may be any type of communications network, such as for example, a traditional circuit switched network (e.g., a public switched telephone network (PSTN)) or a packet network such as an Internet Protocol (IP) network (e.g., an IP Multimedia Subsystem (IMS) network), an asynchronous transfer mode (ATM) network, a wireless network, a cellular network (e.g., 2G, 3G, and the like), a long term evolution (LTE) network, and the like related to the current disclosure. It should be noted that an IP network is broadly defined as a network that uses Internet Protocol to exchange data packets.

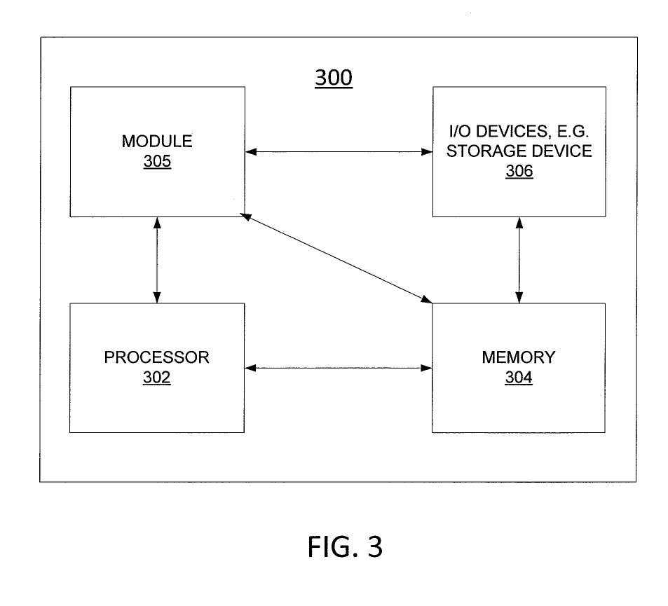

In one embodiment, the communications network 101 may include a core network. The core network may include the application server (AS) 118 and the database (DB) 117. The AS 118 may be deployed as a hardware device embodied as a general purpose computer (e.g., the general purpose computer 300 as illustrated in FIG. 3). In one embodiment, the AS 118 may perform the methods and functions described herein (e.g., the method 200 discussed below).

In one embodiment, the communications network 101 may include one or more access networks (e.g., a cellular network, a wireless network, a wireless-fidelity (Wi-Fi) network, a PSTN network, an IP network, and the like) that are not shown to simplify FIG. 1. In one embodiment, the communications network 101 in FIG. 1 is simplified and it should be noted that the communications network 101 may also include additional network elements (not shown), such as for example, border elements, gateways, firewalls, routers, switches, call control elements, various application servers, and the like.

In one embodiment, the database 117 is used for storing ambient information received from various sensor devices and/or sources, TRM sensing data, location of patients, physiological reactions of patients to various environmental conditions, etc. In one embodiment, the database 117 comprises a database component 121 for storing ambient information, a database component 122 for storing TRM sensing data, a database component 123 for storing locations of patients, and a database component 124 for storing physiological reactions of patients to various environmental conditions.

The TRM device 116 may comprise one or more sensor devices for measuring one or more patient physiological conditions. For example, a patient's physical condition that is measured may be the patient's blood pressure, blood glucose level, heart rate, body temperature, skin moisture, respiratory rhythm, electrocardiogram of the heart, gaiting (e.g., walking, running, falling, standing, lying down, sitting and the like) etc. The term “patient” should be broadly interpreted as a “monitored individual”, and does not require the “monitored individual” to be sick, or diagnosed with a disease.

The ambient data collection devices 111-115 comprise a variety of ambient sensor devices that are used for gathering ambient information from a patient's surrounding. For example, a device may comprise a sensor device for measuring temperature (e.g., a thermometer), a sensor device for measuring humidity (e.g., a hygrometer), a sensor device for measuring ambient noise level (e.g., a sensor having a microphone), a sensor device for measuring electromagnetic radiation levels (e.g., an electromagnetic wave detection sensor or meter), a sensor device for measuring a light intensity level (e.g., a sensor having one or more photodiodes that indicate levels of brightness) and the like.

The server 118 comprises an application server for providing an ambient augmented TRM method in accordance with the present disclosure as described below. In one embodiment, the server 118 performs one or more steps of the method 200 as described below.

It should be noted that the above illustrated network 100 is only illustrative and the number of components or elements are not specifically limited as shown. Any number of components or elements can be deployed.

In one embodiment, a health monitoring system may monitor the health of patients. For example, the health of patients may be monitored and analyzed via a generic analytic engine. One approach for determining whether there is a need for sending an alert is comparing data obtained for a patient, against either historical norms established for the patient or historical norms established for the general population. For example, a patient's doctor or nurse (broadly a health care provider) may receive alerts as to abnormal physiological conditions of the patient. The physiological condition is deemed abnormal based on comparisons against various norms.

However, the decision for sending an alert to the health care provider may be inaccurate. For example, if the patient is uncomfortable because he/she is located in a hot and noisy environment, e.g., in a crowded room that is not air conditioned, etc., the patient may experience a physiological reaction such as an increase in blood pressure. When the doctor is alerted, the doctor may simply instruct the patient to take more medication based purely on the monitored data reported for the patient. Although the patient received the instruction to take more medication, a simple change in the environment may actually be an adequate intervention. Moreover, the alert may even be unnecessary in view of the environment in which the patient is located. Thus, the correlation of the physiological response of the patient to the immediate surrounding environment will provide a more accurate medical assessment, thereby leading to the issuance of more appropriate corrective actions for the patient to take in light of the specific environment.

In one embodiment, the method of the present disclosure provides ambient augmented telehealth remote monitoring. An alert for a patient is assessed in view of ambient information that is gathered near the vicinity of the patient. The method then provides a report to pertinent recipients (e.g., a health care provider, a guardian and the like) based on the assessment.

First, the method collects data on physiological conditions of a patient. For example, the method may collect the data on the physiological conditions of the patient from a TRM device 116. The TRM device 116 converts the patient's physiological condition into data and sends the data remotely to the database 117, e.g., via wireless communications. The method then stores the information in the database 117.

Similarly, the method collects data on ambient information in the vicinity of the patient. For example, the method may collect the data on the ambient information via ambient data collection devices 111-115. The ambient data collection devices 111-115 send the ambient data to the database 117 wirelessly for storage, e.g., via wireless communications.

In one embodiment, an ambient sensor device comprises a sensor device that is under the control of the patient. In one embodiment, the sensor device that is under the control of the patient is a sensor device to be carried by the patient. For example, the device that is under the control of the patient is a device to be worn by the patient or carried by the patient.

In one embodiment, an ambient sensor device comprises a device that is embedded (e.g., deployed on a wall, a ceiling, a floor, a piece of furniture and the like) in an environment in the vicinity of the patient. For example, the ambient sensor device that is embedded in the environment may be owned and/or managed by a third party, e.g., a landlord of a building, a government entity, and so on. For example, a plurality of ambient sensor devices can be deployed throughout an office building, a residential home, an apartment, a warehouse, a store, a stadium, a metro or subway station, a public park and the like. Each of the ambient sensor devices may have the ability to be uniquely identified, e.g., an IP address, a unique code or a unique location, e.g., Global Positioning System (GPS) coordinates and the like. Furthermore, each of the ambient sensor devices can be implemented with communications capability, e.g., a transceiver, a transmitter, a receiver, an antenna and the like.

If the ambient sensor device comprises a sensor device that is managed by a third party, the method of the present disclosure gathers the ambient information from the patient's surrounding, by first obtaining a location of the patient, and retrieving the ambient information based on the location of the patient. For example, ambient sensor information may be continuously gathered from various sources and stored in a database. When the patient's location is received or determined, the method may then use the patient's location to identify and retrieve the applicable ambient information from the database. For example, the patient may be at a work location where ambient sensor devices are deployed throughout the work location, e.g., a large mail processing center. Under this example, ambient information such as noise levels, temperature, and humidity can be monitored throughout the entire mail processing center.

If an alert is received for a patient, the method analyzes the TRM data of the patient in view of the ambient information in the vicinity of the patient. The method then provides a report in accordance with the analysis. The report can be sent to the patient, a doctor, a nurse, an emergency service provider, a care giver, a guardian, a contact provided by the patient (e.g. a family member, a friend, a neighbor, etc.). In one embodiment, the server provides an appropriate follow-up instruction in accordance with the ambient augmented TRM. For example, the server may provide an instruction to the patient as to avoiding a noisy environment. In another example, the server may provide an instruction to the patient as to consuming more fluids. In another example, the server may provide an instruction to the patient as to contacting a caregiver.

In one embodiment, the method stores in a database a physiological condition (e.g., a reaction or response) associated with an ambient condition. For example, a patient may have a physiological reaction in response to a high temperature (e.g., a physiological reaction comprising an elevated body temperature), a sudden loud audio stimulation (e.g., a physiological reaction comprising a sudden increase in heart rate), a sudden bright or high intensity visual stimulation (e.g., a physiological reaction comprising blinking of the eyes or gaiting that indicates stumbling), a high humidity level (e.g., a physiological reaction comprising a sudden increase in sweating), presence of airborne contaminants that produce breathing difficulty (volatile organic compounds or biological suspensions such as pollen), etc. A knowledge database may then be built for associating physiological reactions of the patient with various environmental conditions. For example, over a long duration of time, the knowledge database may enable determination of whether a patient's blood pressure is affected by one or more of the above environmental stimulations. In another example, the knowledge database may enable determination of whether a time of day affects a physiological reaction. In yet another example, a patient may have a pacemaker or similar device which may be affected by electromagnetic signals in the vicinity of the patient.

In one embodiment, the method stores in the database a level of physiological reaction association with the ambient condition. For example, the reaction may be considered a mild reaction, a severe reaction, etc. A patient may then be able to receive an instruction on avoiding certain trigger environments to reduce certain physiological reactions that may be deemed unhealthy to the patient. For example, a patient may be instructed to avoid noisy environments.

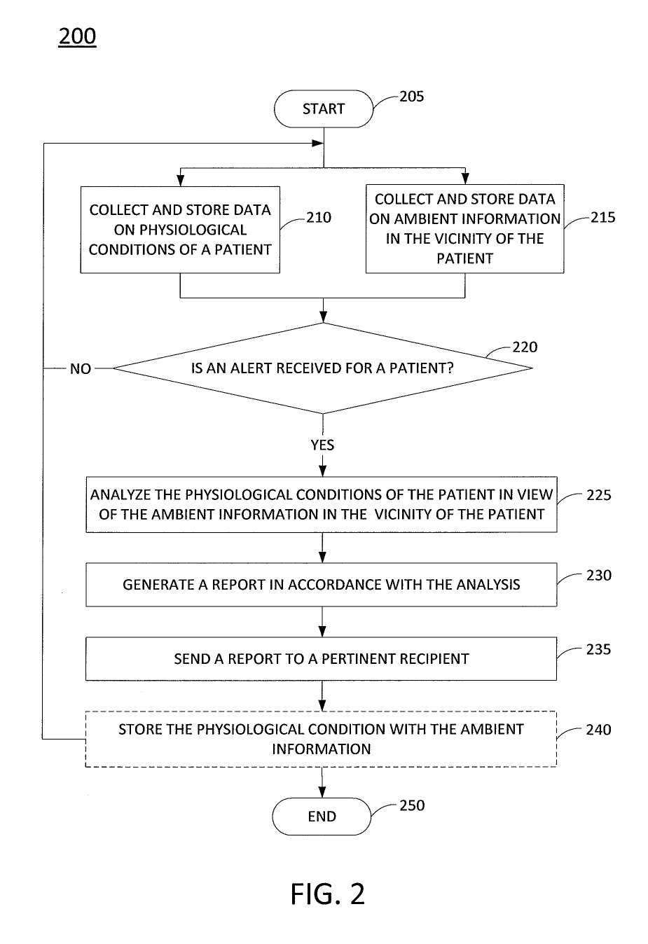

FIG. 2 illustrates a flowchart of a method 200 for providing an ambient augmented telehealth remote monitoring. In one embodiment, the method may be implemented in a server, e.g., an application server 118 for providing an ambient augmented telehealth remote monitoring service or the general purpose computer as described in FIG. 3. Method 200 starts in step 205 and proceeds to steps 210 and 215.

In step 210, method 200 collects and stores data on physiological conditions of a patient. For example, the method may collect the data on the physiological conditions via a TRM device.

In step 215, method 200 collects and stores data on ambient information in the vicinity of the patient. For example, the method may collect the data on the ambient information via ambient data collection devices or from third parties responsible for managing the environment near the patient.

In step 220, method 200 determines if an alert is received for a patient. If an alert is received for a patient, the method proceeds to step 225. Otherwise, the method proceeds to steps 210 and 215. The alert may be generated when a threshold correlating to a particular physiological condition of the patient has been reached, e.g., a body temperature of 100° F., a heart rate of 120 beats per minute, a blood pressure level of 130 over 80 and the like.

In step 225, method 200 analyzes the physiological conditions of the patient in view of the ambient information in the vicinity of the patient. For example, the method analyzes the ambient information (broadly assesses the impact of the ambient information) to determine whether there is an environment reason that may have caused the alert to be generated. In other words, the method attempts to identify and correlate an environment cause as the trigger for the physiological reaction. For example, if the patient's heart rate suddenly surges, and the ambient information indicates that there was a sudden loud noise at a similar time frame when the patient experienced the sudden heart rate surge, the method may conclude that the alert associated with the sudden heart rate surge was caused by a sudden change in the surrounding environment. In one embodiment, data pertaining to the physiological conditions and data pertaining to the ambient information are collected and analyzed over a period of time, e.g., over 5 minutes, 15 minutes, over one hour, over a day, and the like. This allows method 200 to determine whether the physiological condition is transient or persistent, thereby leading to a more accurate assessment.

In step 230, method 200 generates a report in accordance with the analysis. For example, the method may generate a report indicating that the alert was caused by environment conditions. Alternatively, the method 200 may generate a report indicating that the alert was not caused by environment conditions. The report may optionally include a recommendation for a corrective action to be taken, e.g., instruction for the patient to leave the immediate location, to call a doctor, to drink more fluids, to lie down, to take a medication, and the like.

In step 235, method 200 sends a report to a pertinent recipient. The pertinent recipient may be the patient, a doctor, an emergency service provider, a care giver, a guardian, a contact provided by the patient (e.g. a family member, a friend, a neighbor, etc.).

In optional step 240, method 200 associates and stores the physiological condition with the ambient information. A knowledge database may then be built for associating physiological conditions of the patient with ambient information. In one embodiment, the method also stores a level of the physiological condition. For example, the condition may be considered a mild condition, a severe reaction, etc. A numbering system can be used, e.g., ranging from 1-10, where “1” represents a mild condition and “10” represents a severe condition. The method then either proceeds to step 250 to end processing the current alert, or to steps 210 and 215.

The GAE can also record instantaneous, short-term, medium-term and long-term measurement trends in separate data storage areas. These data can be used to generate histograms of events and severities and to subsequently infer behaviors that may evolve over time (such as decreased mobility, disturbed sleep habits, or depression for example.

FIG. 3 depicts a high-level block diagram of a general-purpose computer suitable for use in performing the functions described herein. As depicted in FIG. 3, the system 300 comprises one or more hardware processor elements 302 (e.g., a central processing unit (CPU), a microprocessor, or a multi-core processor), a memory 304, e.g., random access memory (RAM) and/or read only memory (ROM), a module 305 for providing ambient augmented telehealth remote monitoring, and various input/output devices 306 (e.g., storage devices, including but not limited to, a tape drive, a floppy drive, a hard disk drive or a compact disk drive, a receiver, a transmitter, a speaker, a display, a speech synthesizer, an output port, an input port and a user input device (such as a keyboard, a keypad, a mouse, a microphone and the like)).

Although only one processor element is shown, it should be noted that the general-purpose computer may employ a plurality of processor elements. Furthermore, although only one general-purpose computer is shown in the figure, if the method(s) as discussed above is implemented in a distributed or parallel manner for a particular illustrative example, i.e., the steps of the above method(s) or the entire method(s) are implemented across multiple or parallel general-purpose computers, then the general-purpose computer of this figure is intended to represent each of those multiple general-purpose computers. Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented.

It should be noted that the present disclosure can be implemented in software and/or in a combination of software and hardware, e.g., using application specific integrated circuits (ASIC), a programmable logic array (PLA), including a field-programmable gate array (FPGA), or a state machine deployed on a hardware device, a general purpose computer or any other hardware equivalents, e.g., computer readable instructions pertaining to the method(s) discussed above can be used to configure a hardware processor to perform the steps, functions and/or operations of the above disclosed methods. In one embodiment, instructions and data for the present module or process 305 for providing ambient augmented telehealth remote monitoring (e.g., a software program comprising computer-executable instructions) can be loaded into memory 304 and executed by hardware processor element 302 to implement the steps, functions or operations as discussed above in connection with the exemplary method 200. Furthermore, when a hardware processor executes instructions to perform “operations”, this could include the hardware processor performing the operations directly and/or facilitating, directing, or cooperating with another hardware device or component (e.g., a co-processor and the like) to perform the operations.

The processor executing the computer readable or software instructions relating to the above described method(s) can be perceived as a programmed processor or a specialized processor. As such, the present module 305 for providing ambient augmented telehealth remote monitoring (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. More specifically, the computer-readable storage device may comprise any physical devices that provide the ability to store information such as data and/or instructions to be accessed by a processor or a computing device such as a computer or an application server.

While various embodiments have been described above, it should be understood that they have been presented by way of example only, and not a limitation. Thus, the breadth and scope of a preferred embodiment should not be limited by any of the above-described exemplary embodiments, but should be defined only in accordance with the following claims and their equivalents.

What is claimed is:

1. A method for processing an alert, comprising:
receiving, by a processor, the alert reporting a physiological condition for an individual;
analyzing, by the processor, the physiological condition of the individual in view of ambient information in a vicinity of the individual;
generating, by the processor, a report in accordance with the analyzing; and
sending, by the processor, the report to a recipient.

2. The method of claim 1, further comprising:
associating the physiological condition with the ambient information.

3. The method of claim 2, further comprising:
storing a level of the physiological condition.

4. The method of claim 1, wherein data of the physiological condition of the individual is received via a portable monitoring device worn by the individual.

5. The method of claim 4, wherein the portable monitoring device comprises a device for measuring at least one of: a blood pressure, a blood glucose level, a heart rate, a body temperature, a respiratory rhythm, an electrocardiogram, and a gaiting.

6. The method of claim 1, wherein data of the ambient information is received via an ambient data collection device.

7. The method of claim 6, wherein the ambient data collection device comprises a device for measuring at least one of: a temperature level, a humidity level, an ambient noise level, a light intensity level, and an electromagnetic radiation level.

8. The method of claim 6, wherein the ambient data collection device comprises a device that is under a control of the individual.

9. The method of claim 8, wherein the device that is under the control of the individual is a device for being carried by the individual.

10. The method of claim 8, wherein the device that is under the control of the individual is a device for being worn by the individual.

11. The method of claim 6, wherein the ambient data collection device comprises a device that is embedded in an environment in the vicinity of the individual.

12. The method of claim 11, wherein the device that is embedded in the environment is managed by a third party entity.

13. The method of claim 12, wherein the analyzing uses the ambient information that is based on a location of the individual.

14. The method of claim 1, wherein the recipient comprises the individual.

15. The method of claim 1, wherein the recipient comprises a doctor of the individual.

16. The method of claim 1, wherein the recipient comprises a contact provided by the individual.

17. The method of claim 1, wherein the report comprises an instruction to leave the vicinity.

18. The method of claim 1, further comprising:
collecting data on the physiological condition of the individual over a period of time; and
collecting data on the ambient information in the vicinity of the individual over the period of time.

19. A computer-readable storage device storing a plurality of instructions which, when executed by a processor, cause the processor to perform operations for processing an alert, the operations comprising:
receiving the alert reporting a physiological condition for a individual;
analyzing the physiological condition of the individual in view of ambient information in a vicinity of the individual;
generating a report in accordance with the analyzing; and
sending the report to a recipient.

20. An apparatus for processing an alert, comprising:
a processor; and
a computer-readable storage device storing a plurality of instructions which, when executed by the processor, cause the processor to perform operations, the operations comprising:
receiving the alert reporting a physiological condition for a individual;
analyzing the physiological condition of the individual in view of ambient information in a vicinity of the individual;
generating a report in accordance with the analyzing; and
sending the report to a recipient.
